(19) United States
(12) Patent Application Publication
(10) Pub. No.: US 2024/0299316 A1
(43) Pub. Date: Sep. 12, 2024

(54) METHOD FOR TREATING CANCER WITH ACYLFULVENE AND RADIATION

(71) Applicant: LANTERN PHARMA INC., Dallas, TX (US)

(72) Inventor: Kishor Bhatia, Ellicott, MD (US)

(73) Assignee: LANTERN PHARMA INC., Dallas, TX (US)

(21) Appl. No.: 18/669,238

(22) Filed: May 20, 2024

Related U.S. Application Data
(63) Continuation of application No. PCT/US22/80150, filed on Nov. 18, 2022.
(60) Provisional application No. 63/264,290, filed on Nov. 18, 2021.

(51) Int. Cl. A61K 31/122; A61K 31/282; A61K 31/337; A61K 31/513; A61K 31/704; A61K 31/7068; A61N 5/10; A61P 35/00

(52) U.S. Cl. CPC A61K 31/122; A61K 31/282; A61K 31/337; A61K 31/513; A61K 31/704; A61K 31/7068; A61N 5/1001; A61P 35/00; A61N 2005/1087

(57) ABSTRACT

The methods include treating cancer by the administration of an effective amount of acylfulvene to a subject in need thereof and an effective amount of radiation. The irradiation occurs before or concurrently with the administration of the effective amount of acylfulvene.

### CROSS-REFERENCE TO RELATED APPLICATION

This application is a continuation of International Patent Application No. PCT/US2022/80150, which claims the benefit of U.S. Provisional Patent Application No. 63/264,290 filed Nov. 18, 2022, each of which is incorporated by reference herein in its entirety.

### TECHNICAL FIELD

This application relates generally to the field of chemistry and oncology. More particularly, this application relates to methods for treating solid tumors using acylfulvene (e.g., hydroxyureamethyl acylfulvene) and radiation.

### BACKGROUND

Cancer is one of the most common causes of death in people. The development of therapeutic strategies for patients with advanced cancer has markedly improved overall survival. However, resistance to anticancer reagents is inevitable, and the prognosis of advanced cancer remains poor. There are several potential sources of cancer drug resistance, including alterations to drug transporters, the suppression of apoptosis, mitochondrial alterations, the promotion of DNA damage repair, autophagy, epithelial-mesenchymal transition, and cancer stem cells (CSCs). Appropriate strategies that consider the mechanisms are necessary to cure cancer.

Radiation therapy (also called radiotherapy) is a cancer treatment that uses high doses of radiation to kill cancer cells and shrink tumors. At high doses, radiation therapy kills cancer cells or slows their growth by damaging their DNA. Cancer cells whose DNA is damaged beyond repair stop dividing or die. When the damaged cells die, they are broken down and removed by the body. Radiation is used to produce ionizing reactions that form free radicals, which react with DNA, and RNA triggering programmed cell death (apoptosis) in cancer cells.

Combination-therapy treatments for cancer have become more common, in part due to the perceived advantage of attacking the disease via multiple avenues. For example, Surgery or radiation therapy treats cancer that is confined locally, while cancer drugs also kill the cancer cells that have spread to distant sites. Although many effective combination-therapy treatments have been identified over the past few decades; in view of the continuing high number of deaths each year resulting from cancer, a continuing need exists to identify effective therapeutic regimens for use in anticancer treatments.

Accordingly, there is always a need for improved methods to treat cancer.

### SUMMARY

This application discloses a method or a combination therapy for treating solid cancers or tumors using radiation and acylfulvene. One aspect includes a method of treating cancer in a subject including administering to the subject acylfulvene; and radiation, in an amount effective to treat, wherein the subject is identified as having cancer cells that express an elevated level of PTGR 1 relative to a reference level after treatment with the effective amount of the radiation. The radiation can be administered before the administration or dose of acylfulvene or at the beginning of a treatment regimen including acylfulvene. The subject has an increased expression of PTGR1 relative to a reference level after the effective amount of radiation but prior to the administration of the hydroxyureamethyl acylfulvene. The acylfulvene can be (−)-hydroxyureamethyl acylfulvene or Irofulven. The cancer may be resistant to chemotherapy or resistant to (−)-hydroxyureamethyl acylfulvene.

Another aspect includes a method in which the treated cancer is a solid tumor.

Another aspect includes a method in which the subject is diagnosed with a pancreatic cancer, a lung cancer, a breast cancer, a colon cancer, a liver cancer, a skin cancer, a brain cancer, a kidney cancer, an ovarian cancer, a uterine cancer, a prostate cancer, or a brain cancer.

Another aspect includes a method in which the cancer or cancer cells exposed to the radiation express additional levels or higher levels of PTGR1 (than prior to exposure with the radiation).

Another aspect includes a method in which the radiation is administered to the subject before the acylfulvene or hydroxyureamethyl acylfulvene (or additional dose of the same) is administered to the subject.

Another aspect includes a method in which the tumors are selected from the group consisting of a squamous cell carcinoma tumor (SCC tumor), a pancreatic carcinoma tumor and a colon carcinoma tumor.

Another aspect includes a method of treating cancer or tumor in a subject including administering a therapeutically effective amount of hydroxyureamethyl acylfulvene to a subject in need thereof and irradiating the cancer or tumor with a therapeutically effective dose of radiation, wherein the irradiation occurs before or concurrently with the administration of the effective amount of the hydroxyureamethyl acylfulvene. The tumor can have an increased expression of PTGR1 relative to a reference level after treatment with the effective amount of radiation. The tumor may be in an organ selected from the group consisting of breast, lung, brain, liver, skin, kidney, GI organ, prostate, bladder, brain and gynecological organ. The radiation therapy can be selected from irradiation, fractionated radiotherapy, radio surgery, and a combination thereof. The subject can be an animal or mammal.

Another aspect includes a method in which the tumor or cancer has an increased expression of PTGR1 relative to a reference level after the effective amount prior to administration of the hydroxyurcamethyl acylfulvene.

Another aspect includes a method of treating solid tumors in a patient including administering to the patient: one or more or a plurality of doses of (−)-hydroxyureamethyl acylfulvene or acylfulvene; and radiation, in an amount effective to treat. The subject is identified as having cancer cells that express an elevated level of PTGR1 relative to a reference level after treatment with the effective amount of the radiation; and the radiation is administered to the patient prior to administering (−)-hydroxyureamethyl acylfulvene. The radiation can be applied after before each dose in a treatment cycle or intermittently with the doses of the plurality of doses.

### BRIEF DESCRIPTION OF THE DRAWINGS

FIG. 1 shows that radiation and hydroxyureamethyl acylfulvene had synergy in shrinking tumors in Panc03.27 xenografts or cells;

FIG. 2 shows terminal tumors at day 21 from group treated with radiation (RT) and hydroxyurcamethyl acylfulvene are statistically smaller (p-value 0.017) than those only treated with either hydroxyureamethyl acylfulvene alone or radiation alone; and

FIG. 3 shows excised terminal tumors from the various treatment arms.

### Definitions

Unless defined otherwise, all technical and scientific terms used herein have the same meaning as is commonly understood by one of skill in the art to which the subject matter herein belongs. As used herein, the following definitions are supplied in order to facilitate the understanding of the present invention.

The terms “administer”, “administering” or “administered” refer to the act of giving an agent or therapeutic treatment to a physiological system (e.g., a subject or in vivo, in vitro, or ex vivo cells, tissues, and organs).

The term “effective amount” as used herein refers to the amount of an agent needed to alleviate at least one or more symptoms of the disease or disorder, and relates to a sufficient amount of pharmacological composition to provide the desired effect. The term “therapeutically effective amount” therefore refers to an amount of the agent that is sufficient to provide a particular effect when administered to a typical subject. An effective amount may be an amount sufficient to decrease the symptoms of a disease responsive to the combination of radiation and acylfulvene (e.g., (−)-hydroxyureamethyl acylfulvene). For cancer therapy, efficacy in vivo can, for example, be measured by assessing the duration of survival, time to disease progression (TTP), the response rates (RR), duration of response, and/or quality of life. Effective amounts may vary, as recognized by those skilled in the art, depending on route of administration, excipient usage, and co-usage with other agents. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

The terms “patient,” “subject,” “individual,” and “host” refer to either a human or a non-human animal suffering from or suspected of suffering from a disease or disorder associated with aberrant biological or cell growth activity.

The term “treat” is used and includes both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

The term “preventing” when used in relation to a condition or disease such as cancer, refers to a reduction in the frequency of, or delay in the onset of, symptoms of the condition or disease. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The terms “expression level” and “level of expression,” as used herein, refer to the amount of a gene product in a cell, tissue, biological sample, organism, or patient, e.g., amounts of DNA, RNA (e.g. messenger RNA (mRNA)), or proteins corresponding to a given gene.

The term “electromagnetic radiation (wave)” or “electromagnetic radiation (wave)” refers to radiation (wave) with electrical and magnetic component which includes (but not limited to) optical (ultraviolet, visible, and infrared light), microwave, and radiofrequency radiation (wave).

The term “pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

The term “similar dose of ionizing radiation” refers to a dose of ionizing radiation that is identical to, nearly the same, or substantially the same as the effective dose administered to a tumor in another subject, or administered to a tumor in the same subject undergoing an existing course of treatment. The term encompasses the normal and expected variation in ionizing radiation doses delivered by a medical technician skilled in the art of administering ionizing radiation to a tumor in a subject. For example, the term encompasses variation in the effective dose administered to a tumor of less than 10%, less than 5%, or less than 1%. The subject can be a human or non-human animal, such as a companion animal (e.g., cat, dog) or farm animal (e.g., cow, horse, etc.).

As used herein, the term “selectively” means tending to occur at a higher frequency in one population than in another population. The compared populations can be cell populations. Preferably, a compound of the present invention, or a pharmaceutically acceptable salt or solvate thereof, acts selectively on a cancer or precancerous cell but not on a normal cell. Preferably, a compound of the present invention, or a pharmaceutically acceptable salt or solvate thereof, acts selectively to modulate one molecular target (e.g., nucleotide excision repair (NER) players ERCC3 or PTGR1 (Prostaglandin Reductase 1)). The invention also provides a method for selectively inhibiting the activity of an enzyme, such as NER proteins. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

“Refractory or resistant cancer” means cancer that does not respond to treatment. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments, the prior therapy is monotherapy. In certain embodiments, the prior therapy is combination therapy.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder. In some embodiments, the therapeutically effective amount of acylfulvene or a pharmaceutically acceptable salt thereof is selected from the group consisting of 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day. In some embodiments, the therapeutically effective amount of hydroxyureamethyl-acylfulvene or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day.

### DETAILED DESCRIPTION

This application provides a combination therapy for treating solid cancers using radiation and acylfulvene. In some embodiments, the combination therapy can be used to treat occurrence or recurrence of solid cancers (e.g., lung cancer, breast cancer, prostate cancer, colon cancer, rectum cancer, and bladder cancer), glioblastoma and atypical teratoid rhabdoid, and renal cell carcinoma). In other embodiments, the therapy includes a combination therapy that can be used to treat biochemical occurrence and recurrence of blood cancers in which an acylfulvene (e.g., hydroxyureamethyl acylfulvene) or salt thereof and a radiation administered in a therapeutically effective amount to the patient (e.g., before and currently a dose in a treatment cycle). In embodiments, the therapy includes administering a combination of active agents including an illudin or an illudin analog (e.g., acylfulvene) and a radiation. In other embodiments, the therapy includes administering a combination of other therapies. In certain embodiments, treatment includes inhibiting the growth of primary tumor cells, inhibiting the formation of metastases, inhibiting the growth of metastases, killing circulating cancer cells, inhibiting the growth and/or survival of cancer stem cells, inducing remission, extending remission, or inhibiting recurrence. In some embodiments, treatment includes inhibiting the growth and/or survival of cancer stem cells.

### Illudin or Acylfulvene

In one embodiment, this application includes the use of an illudin or illudin analog (e.g., acylfulvene). Acylfulvene is a class of cytotoxic semi-synthetic derivatives of illudin, a natural product that can be extracted from the jack o′lantern mushroom (Omphalotus olearius). Acylfulvene, derived from the sesquiterpene illudin S by treatment with acid (reverse Prins reaction), is far less reactive to thiols than illudin S.

In one example, the acylfulvene is (−)-hydroxyureamethyl acylfulvene (termed LP-184 by Lantern Pharma Inc.), which shifts light negatively, is shown below:

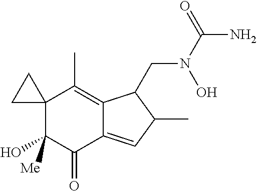 [SMILES: CC1=C2C(=CC(C)C2CN(O)C(N)=O)C(=O)[C@](C)(O)C12CC2]

In another example, the acylfulvene is (+)-hydroxyureamethyl acylfulvene (termed LP-284 by Lantern Pharma Inc.), which shifts light positives, is shown below:

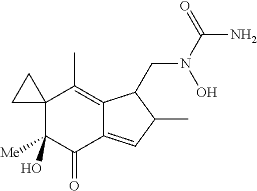 [SMILES: CC1=C2C(=CC(C)C2CN(O)C(N)=O)C(=O)[C@@](C)(O)C12CC2]

(+)-hydroxyurcamethyl acylfulvene and (−)-hydroxyureamethyl acylfulvene are enantiomers and are now known publicly.

In another example, the acylfulvene is Irofulven.

Specific embodiments relate to methods of treating solid tumors or cancer, the methods including the administration of an effective amount of hydroxyureamethyl acylfulvene to a subject in need thereof together with one or more doses of radiation (before, after, or concurrently therewith). Methods are provided for treating a subject with radiation to sensitize the subject to treatment with hydroxyureamethyl acylfulvene. In one example, hydroxyureamethyl acylfulvene can be administered as a monotherapy.

### Radiation

Methods of treating cancer with radiation are well known. Radiation therapy or radiotherapy is the medical use of ionizing radiation, generally as part of cancer treatment to control or kill malignant cells.

The amount of radiation used in photon radiation therapy is measured in gray (Gy), and varies depending on the type and stage of cancer being treated. For curative cases, the typical dose for a solid epithelial tumor ranges from 60 to 80 Gy, while lymphomas are treated with 20 to 40 Gy.

The total dose of radiation is often fractionated (spread out over time) for several important reasons. Fractionation allows normal cells time to recover, while tumor cells are generally less efficient in repair between fractions. Fractionation also allows tumor cells that were in a relatively radio-resistant phase of the cell cycle during one treatment to cycle into a sensitive phase of the cycle before the next fraction is given. Similarly, tumor cells that were chronically or acutely hypoxic (and therefore more radio-resistant) may re-oxygenate between fractions, improving the tumor cell kill.

Fractionation regimens are individualized between different radiation therapy centers and even between individual doctors. In North America, Australia, and Europe, the typical fractionation schedule for adults is 1.8 to 2 Gy per day, five days a week. In some cancer types, prolongation of the fraction schedule over too long can allow for the tumor to begin repopulating, and for these tumor types, including head and neck and cervical squamous cell cancers, radiation treatment is preferably completed within a certain amount of time. For children, a typical fraction size may be 1.5 to 1.8 Gy per day, as smaller fraction sizes are associated with reduced incidence and severity of late-onset side effects in normal tissues.

In some cases, two fractions per day are used near the end of a course of treatment. This schedule, known as a concomitant boost regimen or hyperfractionation, is used on tumors that regenerate more quickly when they are smaller. In particular, tumors in the head-and-neck demonstrate this behavior.

One fractionation schedule that is increasingly being used and continues to be studied is hypofractionation. This is a radiation treatment in which the total dose of radiation is divided into large doses. Typical doses vary significantly by cancer type, from 2.2 Gy/fraction to 20 (y/fraction. The logic behind hypofractionation is to lessen the possibility of the cancer returning by not giving the cells enough time to reproduce and also to exploit the unique biological radiation sensitivity of some tumors. One commonly treated site where there is very good evidence for such treatment is in breast cancer. Short course hypofractionated treatments over 3-4 weeks e.g. 40Gy in 15 fractions or 42.5Gy in 16 fractions, have been shown to be as effective as more protracted 5-6 week treatments with respect to both cancer control and cosmesis (restoration of patient appearance). Those skilled in the art will appreciate the treatment schedules and how to vary the dosage and treatment schedules in combination with the present methods.

Preventive (adjuvant) doses (meaning therapy applied after initial treatment for the cancer) are typically around 45-60 Gy in 1.8-2 Gy fractions (for breast, head, and neck cancers.) Many other factors are considered by radiation oncologists when selecting a dose, including whether the patient is receiving chemotherapy, patient comorbidities, whether radiation therapy is being administered before or after surgery, and the degree of success of surgery.

Delivery parameters of a prescribed dose are determined during treatment planning (part of dosimetry). Treatment planning is generally performed on dedicated computers using specialized treatment planning software. Depending on the radiation delivery method, several angles or sources may be used to sum the total necessary dose. The skilled practitioner designs a plan that delivers a uniform prescription dose to the tumor and minimizes dose to surrounding healthy tissues and side effects.

In one embodiment, the radiation is given in the treatment cycle of acylfulvene or hydroxyureamethyl acylfulvene before or concurrently with each dose. For example, the patient can be radiated and given a dose of acylfulvene or hydroxyureamethyl acylfulvene and then radiated again and given a dose of acylfulvene or hydroxyureamethyl acylfulvene and so forth. The radiation need not be given before any or all doses during a treatment cycle. In some embodiments, the radiation is given some time before the dose of acylfulvene and in other embodiments, the radiation is given some time before the dose of acylfulvene. In yet other embodiments, the dose of acylfulvene may be given after the radiation, but after, the level of PTGR1 is elevated from prior levels. The radiation can be between 3 and 6 Gy, 2 and 4 Gy, 1 and 2 Gy, or 4 and 6 Gy.

In some embodiments, the average and/or ranked expression level of all the biomarkers in the tumor sample is increased or decreased relative to the expression level in normal tissue. Thus, in some embodiments, the average and/or ranked expression level of all the biomarkers in the tumor sample is increased or decreased by at least 10%. 20%, 30%, 40%, 50%, 60%, 70%, 80%, 90% or more compared to the expression level in normal tissue. In some embodiments, the expression levels in normal tissue are normalized to a control or baseline level. It will be understood that the expression level can also be compared to the expression level in the tumor sample before, after or during a treatment, course of treatment, or treatment plan. In one example the biomarker is PTGR1 and is expressed or further expressed as a result of the radiation exposure. In this embodiment, the level of PTGR1 can be measured before or after any dose of radiation.

One embodiment includes co-administering hydroxyureamethyl acylfulvene and an additional therapeutic agent in separate compositions or the same composition. Thus, some embodiments include a first pharmaceutical composition comprising: (a) a safe and therapeutically effective amount of hydroxyureamethyl acylfulvene or pharmaceutically acceptable salts thereof and (b) a second pharmaceutical composition. In some embodiments, the method described herein can further include subjecting the subject to a radiation therapy. In some embodiments, the radiation therapy, which can be administered before, after or concurrently with the administration of hydroxyureamethyl acylfulvene, can be a whole-organ irradiation, fractionated radiotherapy, or radiosurgery.

In some embodiments, the method comprises using a dose threshold to specify limits for the radiation treatment plan, wherein the limits are selected from the group consisting of: a limit on irradiation time for each sub-volume in the target; a limit on irradiation time for each sub-volume outside the target; a limit on dose rate for each sub-volume in the target; and a limit on dose rate for each sub-volume outside the target.

In some embodiments, the dose threshold is dependent on a plurality of biological factors including but not limited to tissue type and/or immunological profile.

In some embodiments, the beams comprise a type of beam selected from the group consisting of: proton; electron; photon; atom nuclei; and ion.

Some embodiments relate to a method of inducing apoptosis in a tumor cell, the method including contacting the tumor cell with hydroxyureamethyl acylfulvene before, after or concurrently with radiation. In some embodiments, the contacting comprises administering an effective amount of hydroxyureamethyl acylfulvene to a subject having the tumor cell before, after or concurrently with radiation.

In another embodiment, the second therapeutic is one or more chemotherapeutic agents selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, cetuximab and a Raf kinase inhibitor.

In another embodiment, the second therapeutic is one or more chemotherapeutic agents selected from paclitaxel or cisplatinum.

The term “combination therapy” can include or includes the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

The methods of combination therapy may or should result in a synergistic effect, wherein the effect of a combination of compounds or other therapeutic agents is greater than the sum of the effects resulting from administration of any of the compounds or other therapeutic agents as single agents. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

Therapeutically effective doses can vary, as recognized by those skilled in the art, depending on the diseases treated, the severity of the disease, the route of administration, the age and general health condition of the patient, excipient usage, the possibility of co-usage with other therapeutic treatments such as use of other agents and the judgment of the treating physician. For example, guidance for selecting an effective dose can be determined by reference to the prescribing information for hydroxyureamethyl acylfulvene or journal discussion of the same.

The dosage ranges for the administration of an agent according to the methods described herein depend upon, for example, the form of the agent, its potency, and the extent to which symptoms, markers, or indicators of a condition described herein are desired to be reduced, for example, the percentage reduction desired for tumor growth. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of an agent described herein in, e.g., the treatment of a condition described herein, or to induce a response as described herein (e.g., solid cancers or blood cancers) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size and/or growth rate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example, treatment of blood cancers in a mouse model. When using an experimental animal model, efficacy of treatment is evidenced when a statistically significant change in a marker is observed, e.g. tumor size and/or growth rate. In some embodiments, the therapeutically effective amount of hydroxyurcamethyl-acylfulvene or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

The administration period can be a multi-week treatment cycle as long as the tumor remains under control and the regimen is clinically tolerated. In some embodiments, a single dosage of hydroxyureamethyl acylfulvene or other therapeutic agent can be administered once a week, and preferably once on each of day 1 and day 8 of a three-week (21 day) treatment cycle. In some embodiments, a single dosage of hydroxyureamethyl acylfulvene or other therapeutic agent can be administered once a week, twice a week, three times per week, four times per week, five times per week, six times per week, or daily during a one-week, two-week, three-week, four-week, or five-week treatment cycle. The administration can be on the same or different day of each week in the treatment cycle.

Another embodiment includes a method of treating solid tumors or cancer in a subject, comprising: (a) irradiating the tumor with radiation; (b) obtaining or having obtained an expression level of the protein by immunohistochemistry, or RNA or the loss of coding regions by FISH, or DNA sequencing in a sample from a subject for a plurality of targets; (c) determining that the subject is sensitive to a treatment with a hydroxyureamethyl acylfulvene; and (d) administering a cancer treatment including hydroxyureamethyl acylfulvene.

Hydroxyureamethyl acylfulvene for use in accordance with the present invention can be mainly administered by parenteral administration, specifically including subcutaneous administration, intramuscular administration, intravenous administration, transcutaneous administration, intrahecal administration, epidural administration, intra joint administration and local administration, or may also be administered in various dosage forms, for example by oral administration if possible.

The injections for parenteral administration include for example sterile, aqueous or non-aqueous solutions, suspensions and emulsions. The aqueous solutions and suspensions include for example distilled water for injections and physiological saline. The non-aqueous solutions and suspensions include for example propylene glycol, polyethylene glycol, vegetable oils such as olive oil, alcohols such as ethanol, and Polysorbate 80 (under trade name). Such composition may contain auxiliary agents such as preservatives, moistening agents, emulsifying agents, dispersing agents, stabilizers (for example, lactose) and dissolution auxiliary agents (for example, meglumine). These are sterilized by filtering through bacteria-retaining filters, blending sterilizing agents, or irradiation. Alternatively, these may be produced once into a sterile solid composition and then dissolved or suspended in sterile water or sterile solvents for injections, prior to use.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Techniques for formulation and administration of the disclosed compounds of the invention can be found in Remington: the Science and Practice of Pharmacy, 19.sup.th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

As used herein, a “subject in need thereof” is a subject having a precancerous condition. Preferably, a subject in need thereof has cancer. A “subject” includes a mammal. The mammal can be e.g., any mammal, e.g., a human, primate, bird, mouse, rat, dog, cat, cow, horse, goat, camel, sheep or a pig. Preferably, the mammal is a human. The subject of the present invention includes any human subject who has been diagnosed with, has symptoms of, or is at risk of developing a cancer or a precancerous condition.

In some embodiments, a subject in need thereof may have a secondary cancer as a result of a previous therapy. “Secondary cancer” means cancer that arises due to or as a result from previous carcinogenic therapies, such as chemotherapy.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer results in a decrease in number and size of tumors. Preferably, after treatment, tumor number or size is reduced by 5% or greater relative to number prior to treatment; more preferably, tumor number or size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt or solvate thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt or solvate thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an arca or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleomorphism.

Administering a composition of the present invention to a cell or a subject in need thereof can result in modulation (i.e., stimulation or inhibition) of an activity of a protein methyltransferase of interest.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc. Natl. Acad. Sci. USA. 100(5): 2674-8, 2003. In an aspect, cell death occurs by apoptosis.

Preferably, an effective amount of a composition of the present invention, or a pharmaceutically acceptable salt or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a composition of the present invention, or a pharmaceutically acceptable salt or solvate thereof, can induce, or activate cell death selectively in cancer cells. Administering to a subject in need thereof a compound of the present invention, or a pharmaceutically acceptable salt or solvate thereof, can induce or activate cell death selectively in cancer cells. Contacting a cell with a composition of the present invention, or a pharmaceutically acceptable salt or solvate thereof, can induce cell death selectively in one or more cells affected by a cell proliferative disorder. Preferably, administering to a subject in need thereof a composition of the present invention, or a pharmaceutically acceptable salt or solvate thereof, induces cell death selectively in one or more cells affected by a cell proliferative disorder.

The present invention relates to a method of treating or preventing cancer by administering a composition of the present invention, or a pharmaceutically acceptable salt or solvate thereof, to a subject in need thereof, where administration of the composition of the present invention, or a pharmaceutically acceptable salt or solvate thereof, results in one or more of the following: prevention of cancer cell proliferation by accumulation of cells in one or more phases of the cell cycle (e.g. G1, G1/S, G2/M), or induction of cell senescence, or promotion of tumor cell differentiation; promotion of cell death in cancer cells via cytotoxicity, necrosis or apoptosis, without a significant amount of cell death in normal cells, antitumor activity in animals with a therapeutic index of at least 2. As used herein, “therapeutic index” is the maximum tolerated dose divided by the efficacious dose.

One skilled in the art may refer to general reference texts for detailed descriptions of known techniques discussed herein or equivalent techniques. These texts can, of course, also be referred to in making or using an aspect of the invention.

### EXAMPLES

A combination therapy approach using post-radiation treatment with (−)-hydroxyureamethyl acylfulvene (or LP-184) was shown to be effective. The treatment strategy included using radiation to elevate expression of PTGR1 in pancreatic cancer. Expression of PTGR1, at least in part, is regulated by nuclear factor erythroid-derived 2 (NRF2), which is a master transcriptional regulator of anti-oxidant response genes and a validated determinant of radioresistance in tumors. NRF2-induced genes neutralize radiation injury and free radicals. PTGR1 is among the NRF2-induced genes and also the exclusive converter of LP-184 to a cytotoxic moiety. Radiation therapy (RT) thus provides an opportunity to increase PTGR1 expression (and linked (−)-hydroxyureamethyl acylfulvene anti-tumor cytotoxicity) selectively in the irradiated tumors.

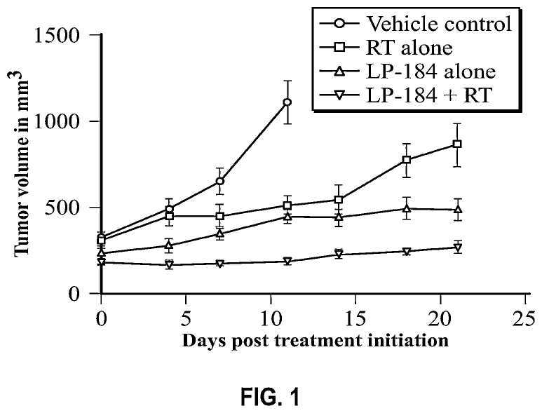

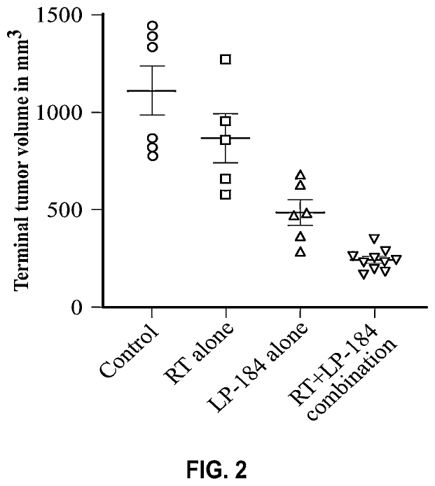

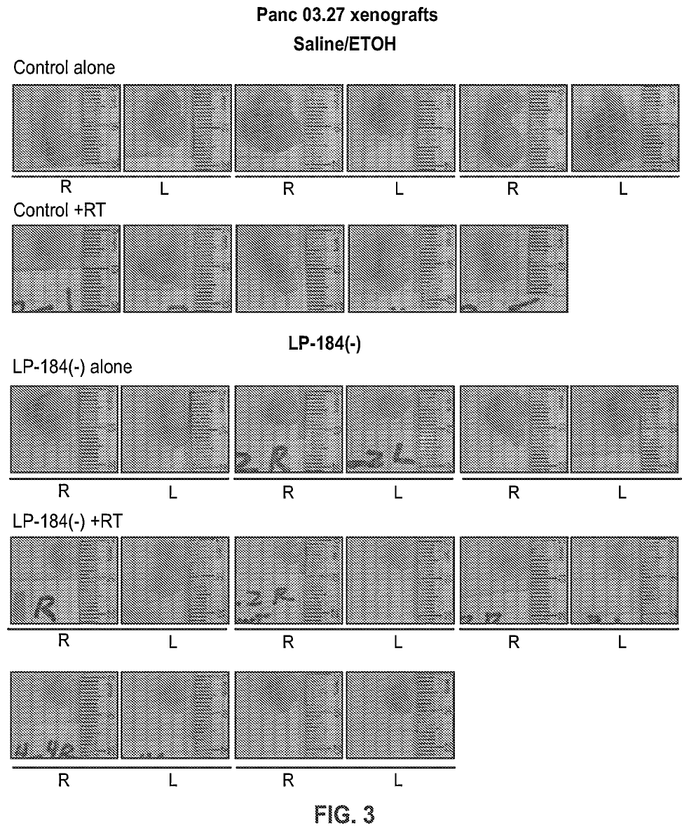

Radiation (RT) pretreatment of pancreatic tumors and subsequent (−)-hydroxyureamethyl acylfulvene treatment in vivo, 4 Gy RT was administered once weekly followed the next day by 3 mg/kg i.p. LP-184 in Panc03.27 xenografts for a total of 3 weeks. Mice bearing pre-established subcutaneous. Panc03.27 xenografts were treated with vehicle control, 4 Gy RT alone, 3 mg/kg LP-184 alone or their combination. The LP-184+RT group showed statistically significant differences in mean terminal tumor volume relative to LP-184 alone or RT alone as shown in FIG. 1. This figure shows the mean tumor volume was about 2 fold (˜1.8 fold) lower in LP-184+RT animals compared to LP-184 alone animals (p=0.017) as displayed in FIG. 2. Excised terminal tumors from the various treatment arms are shown in FIG. 3.

FIG. 1 shows radiation synergized with LP-184 in Panc03.27 xenografts. A line plot of average tumor volume in cubic millimeters over days post treatment initiation in Panc03.27 subcutaneous xenograft tumors treated with vehicle control (gray circles; N=6) is shown, 4 Gy RT alone (red squares; N=5), 3 mg/kg i.p. LP-184 alone (green triangles; N=6), and RT+LP-184 (blue inverted triangles; N=10). Error bars represent SEM.

FIG. 2 shows terminal tumors (day 21) from the RT+LP-184 treatment group are statistically smaller (p value 0.017) than those in the LP-184 alone or RT alone treatment group. FIG. 3 shows a comparison of Panc03.27 xenograft tumors at study termination (day 21) and tumors from the LP-184+RT group appear to have the smallest mean volume.

1. A method of treating cancer in a subject comprising:
(a) administering to the subject acylfulvene; and
(b) locally radiating the subject to increase the expression level of PTGR1 relative to a reference level.

2. A method of claim 1, wherein the acylfulvene is (−)-hydroxyureamethyl acylfulvene.

3. A method of claim 1, wherein the subject has an increased expression of PTGR1 relative to a reference level after the effective amount of radiation but prior to the administration of the hydroxyureamethyl acylfulvene.

4. A method of claim 1, wherein the cancer is a solid tumor.

5. A method of claim 1, wherein the subject is diagnosed with pancreatic cancer, lung cancer, breast cancer, colon cancer, liver cancer, skin cancer, brain cancer, kidney cancer, ovarian cancer, uterine cancer, prostate cancer, or brain cancer.

6. A method of claim 1, further comprising measuring the level of PTGR1 expression in cancer cells from the cancer after the treatment with radiation.

7. A method of claim 1, wherein cells exposed to the radiation express an additional level of PTGR1.

8. A method of claim 1, wherein the radiation is administered to the subject before the hydroxyureamethyl acylfulvene is administered to the subject.

9. A method of claim 1, wherein said tumor is selected from the group consisting of a squamous cell carcinoma tumor (SCC tumor), a pancreatic carcinoma tumor, and a colon carcinoma tumor.

10. A method of claim 1, wherein said chemotherapeutic agent is selected from the group consisting of cisplatin, gemcitabine, 5-fluorouracil (5FU), taxol, and doxorubicin.

11. A method of claim 2, wherein the acylfulvene is (−)-hydroxyureamethyl acylfulvene.

12. A method of claim 1, wherein the cancer is resistant to chemotherapy.

13. A method of claim 1, wherein the cancer is resistant to (−)-hydroxyureamethyl acylfulvene.

14. A method of treating cancer in a subject comprising: (a) administering a therapeutically effective amount of hydroxyureamethyl acylfulvene to a subject in need thereof; and (b) irradiating the tumor with a therapeutically effective dose of radiation, wherein the irradiation occurs before or concurrently with the administration of the effective amount of the hydroxyureamethyl acylfulvene.

15. A method of claim 14, further comprising measuring the level of PTGR1 expression in the cancer after the treatment with radiation.

16. A method of claim 14, wherein the subject has an increased expression of PTGR1 relative to a reference level after the effective amount of radiation.

17. A method of claim 14, wherein the tumor is in an organ selected from the group consisting of breast, lung, brain, liver, skin, kidney, GI organ, prostate, bladder, and gynecological organ.

18. A method of claim 14, wherein the radiation therapy is selected from irradiation, fractionated radiotherapy, radiosurgery, and a combination thereof.

19. A method of claim 14, wherein the subject is an animal.

20. A method of claim 16, wherein the subject has an increased expression of PTGR1 relative to a reference level after the effective amount of radiation but prior to administration of the hydroxyureamethyl acylfulvene.

21. A method of treating solid tumors in a patient comprising:
(a) administering to a subject a plurality of doses of (−)-hydroxyureamethyl acylfulvene; and
(b) radiating the subject, in an amount effective to treat, wherein the subject is identified as having cancer cells that express an elevated level of PTGR1 relative to a reference level after the effective amount of the radiation; the subject is radiated locally to increase expression level of PTGR1 relative to a reference level and the radiation is administered to the patient prior to administering (−)-hydroxyureamethyl acylfulvene and the increased expression of PTGR 1 enhances the therapeutic efficacy of acylfulvene.

22. A method of claim 21, further comprising measuring the level of PTGR1 expression in the cancer after the treatment with radiation.

23. A method of claim 22, wherein the radiation is between 2-5Gy.

24. A method of claim 22, wherein the radiation is between 3-6 Gy.
